(19) United States
(12) Patent Application Publication
(10) Pub. No.: US 2024/0008900 A1
(43) Pub. Date: Jan. 11, 2024

(54) Toe Web File

(71) Applicant: Cherokkee Richardson, Coral Gables, FL (US)

(72) Inventor: Cherokkee Richardson, Coral Gables, FL (US)

(21) Appl. No.: 17/860,088

(22) Filed: Jul. 7, 2022

(51) Int. Cl. A61B 17/54

(52) U.S. Cl. CPC A61B 17/54; A61B 2017/00761

(57) ABSTRACT

This is a slender hollow cylinder shaped pedicure tool with stainless steel micro files designed to manicure one's toe webs by removing dead skin. The top portion contains the stainless steel micro files (which wrap 360 around the cylinder). The top unscrews from the bottom/handle so that accumulated skin shavings can be emptied.

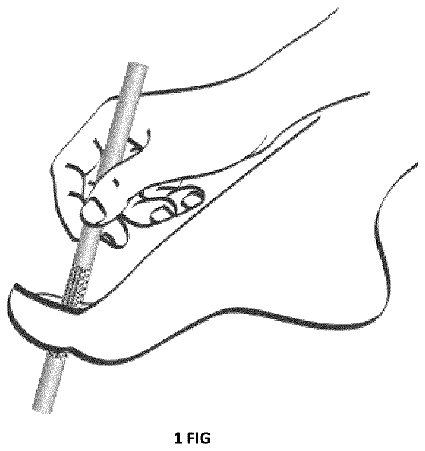

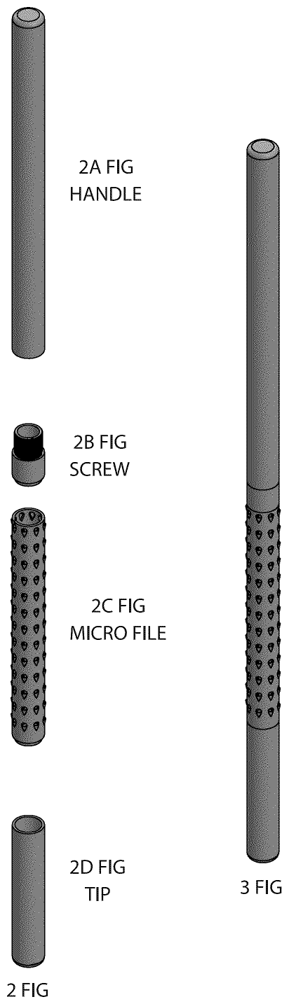

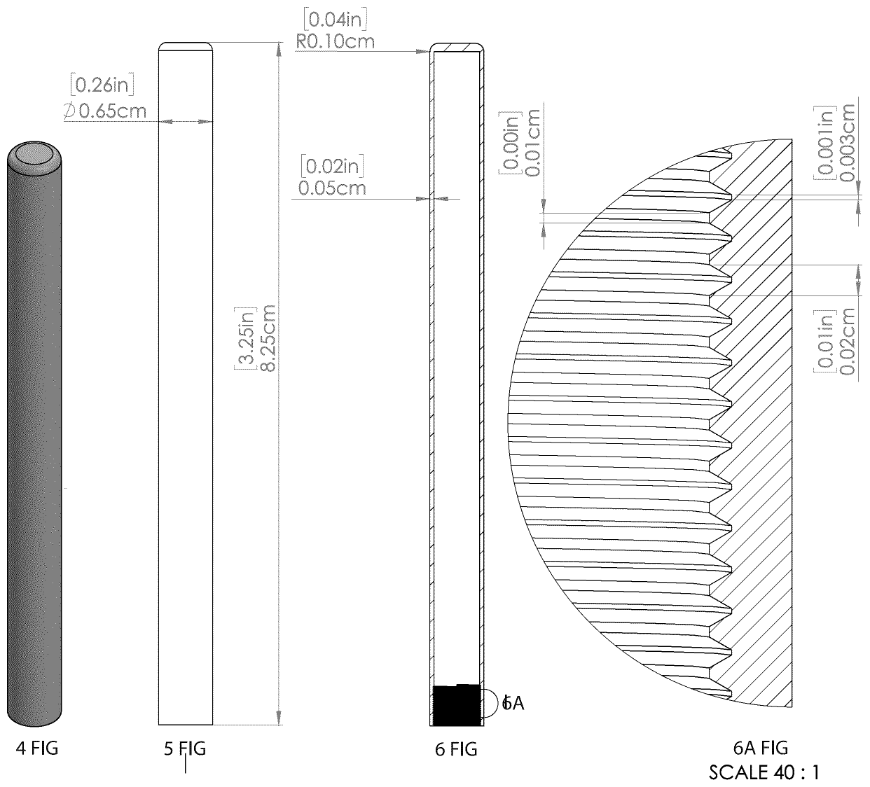

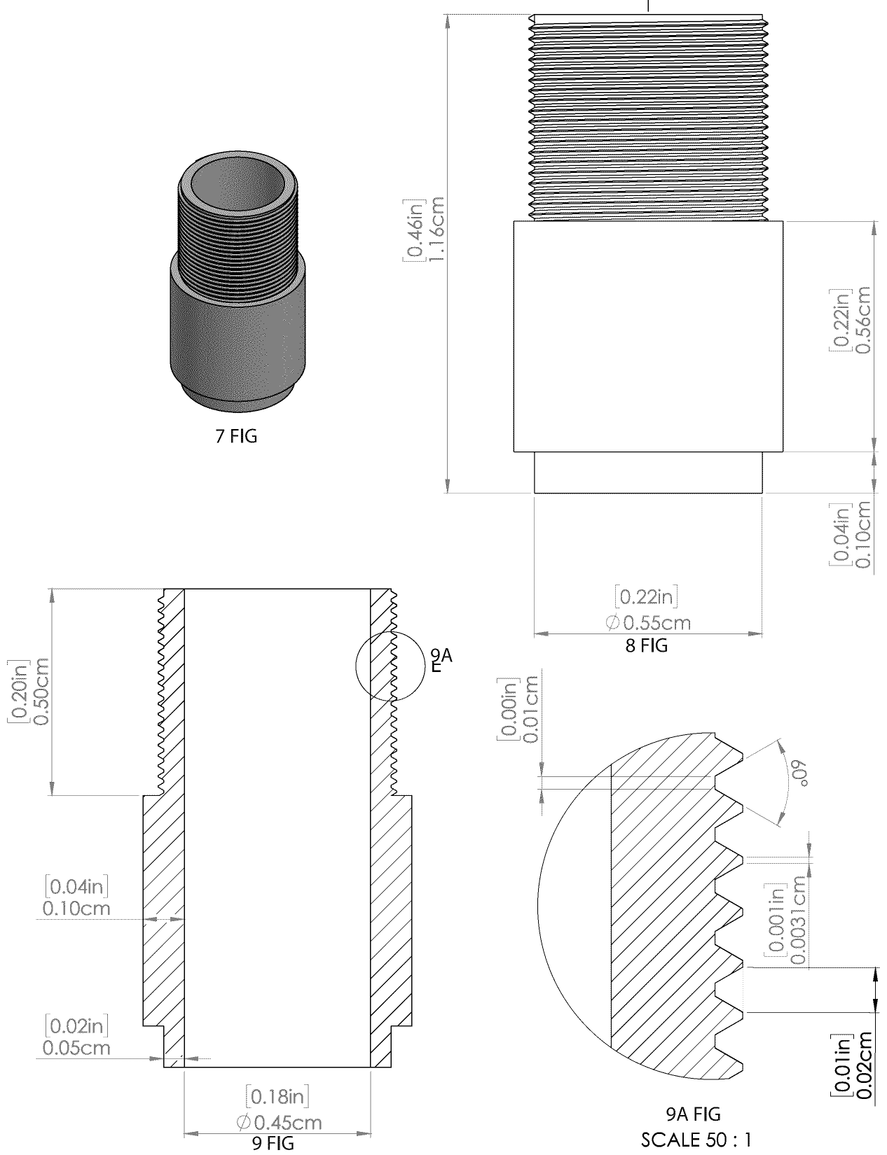

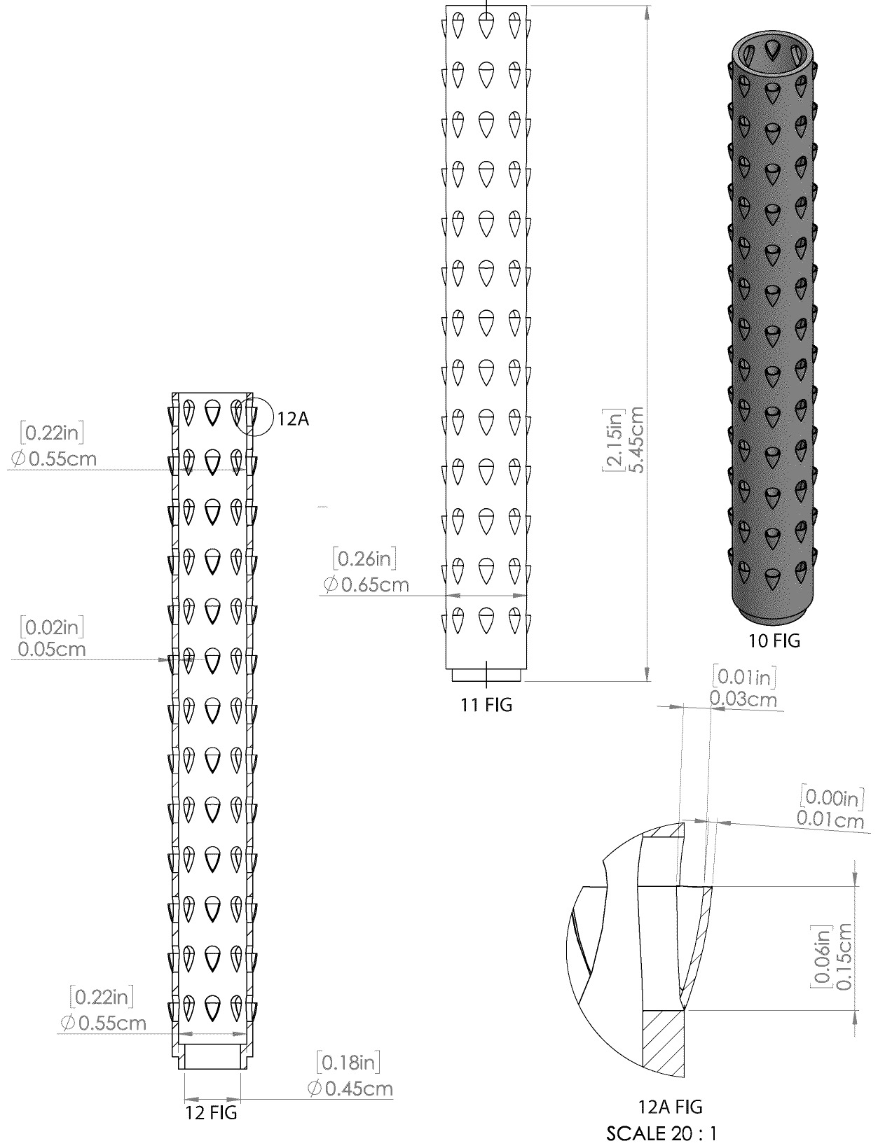

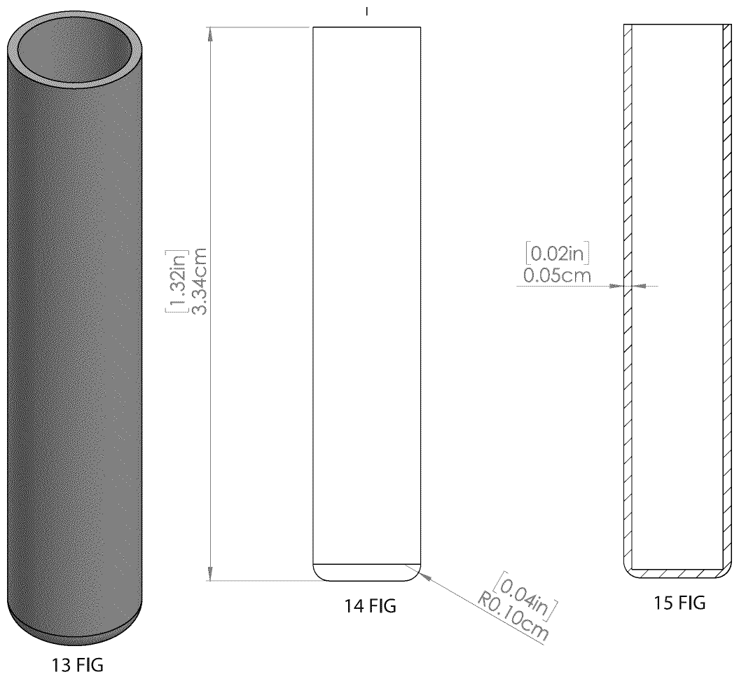

The Toe Web File is a pedicure tool intended to manicure one's toe webs by filing or exfoliating dead skin. The tool has a handle, screw, stainless steel micro files and a tip. The micro files are placed in between one's toes and the handle is used to move the tool in an undulation motion to remove dead skin.

Enclosed are several drawings of the toe web file. The drawings depict the file's application, the alignment of the separate parts and their assembly, and the exterior and interior dimensions of each part.

Drawing 1 FIG. is a 2 dimensional illustration of the toe web file and its application. Drawing 2 FIG. is an exploded view of the assembly of each part (handle (2A), screw (2B), micro files (2C) and tip (2D)). Drawing 3 FIG. is the assembled view of all the separate parts.

Drawing 4 FIG. is an isometric view of the handle. Drawing 5 FIG. illustrates the handle. The part is symmetrical, cylindrical and contains interior threads. Drawing 6 FIG. is the cross section view of 5 FIG. showing the interior details . . . the interior threads and wall thickness. Drawing 6A FIG. shows a scaled view of the thread dimensions.

Drawing 7 FIG. is an isometric view of the screw. Drawing 8 FIG. illustrates a threaded part (screw) that connects the handle to the micro files. The part is symmetrical, cylindrical and has exterior threads. Drawing 9 FIG. is the cross section view of 8 FIG. showing the interior details . . . thread length and wall thickness. Drawing 9A FIG. shows a scaled view of the exterior thread dimensions.

Drawing 10 FIG. is an isometric view of the micro files. Drawing 11 FIG. is an illustration of the micro files. The part is symmetrical, cylindrical and contains multiple protrusions. Drawing 12 FIG. is the cross section view of 11 FIG. showing the interior details . . . wall thickness. Drawing 12A FIG. shows the dimensions of the protrusion on the boundary.

Drawing 13 FIG. is an isometric view of the tip. Drawing 14 FIG. illustrates the tip. The part is symmetrical and cylindrical. Drawing 15 FIG. is the cross section view of 14 FIG. showing the interior details . . . wall thickness.

The micro file portion is made of stainless steel and is hollow. The handle and tip are made of plastic and are hollow allowing for the collection of dead skin flakes. The handle detaches/unscrews so that dead skin flakes can be emptied from the tool. The entire tool is a slender hollow cylinder closed at both ends.

What is claimed is a device/pedicure tool with a cylindrical shape specifically designed to file one's toe webs. This device is composed of a handle, screw, micro files and tip. The screw connects the handle and micro files. The handle and tip contain the closed ends of this hollow cylindrical device. The tip is connected to the micro files. The user holds the device by the handle, places the micro files between the toes and manually moves the device in an undulation motion. This motion and micro files remove dead skin. The screw unscrews for the removal of accumulated skin shavings.

What is claimed is:

1. A device/pedicure tool with a cylindrical shape specifically designed to file one's toe webs.

2. This device is composed of a handle.

3. This device is composed of a screw.

4. This device is composed of a micro files.

5. This device is composed of a tip.

6. The screw connects to the handle.

7. The screw connects to the micro files.

8. The handle contains a closed end of this hollow cylindrical device.

9. The tip contains a closed end of this hollow cylindrical device.

10. The tip is connected to the micro files.

11. The user holds the device by the handle.

12. The user places the micro files between the toes.

13. The user manually moves the device in an undulation motion.

14. The combined undulation motion and micro files remove dead skin.

15. The user unscrews the screw for the removal of accumulated skin shavings.
